(19) United States
(12) Patent Application Publication
(10) Pub. No.: US 2023/0325655 A1
(43) Pub. Date: Oct. 12, 2023

(54) SERVICE AND METHOD FOR BRAIN SEX PHENOTYPE SCORE PREDICTION ON RAW SCALP EEG

(71) Applicants: Mariam KHAYRETDINOVA, Dover, DE (US); Alexey SHOVKUN, Dover, DE (US); Alexey MATVEEV, Dover, DE (US); Andrey KIRYASOV, Dover, DE (US); Maxim POPOV, Dover, DE (US); Ivan MISHANIN, Dover, DE (US)

(72) Inventors: Mariam KHAYRETDINOVA, Dover, DE (US); Alexey SHOVKUN, Dover, DE (US); Alexey MATVEEV, Dover, DE (US); Andrey KIRYASOV, Dover, DE (US); Maxim POPOV, Dover, DE (US); Ivan MISHANIN, Dover, DE (US)

(73) Assignee: Neuroscience Software Inc., Dover, DE (US)

(21) Appl. No.: 17/716,025

(22) Filed: Apr. 8, 2022

(51) Int. Cl. G06N 3/08; G16H 50/20; G06N 3/04; G06V 10/82; G06N 7/00; A61B 5/372; A61B 5/00; G06K 9/62

(52) U.S. Cl. CPC G06N 3/08; G16H 50/20; G06N 3/0454; G06V 10/82; G06N 7/005; A61B 5/372; A61B 5/7267; G06K 9/6267; G06T 2207/20084

(57) ABSTRACT

The present invention relates to methods and systems for brain sex phenotype score prediction according to application of artificial intelligence algorithms to scalp electroencephalography data. Whether male- or female-born, each person does not necessarily abide by biologically predetermined sex for the brain. The human brain is sexually dimorphic, which matters when a decision on mental health and treatment selection is made. A brain sex phenotype score is introduced to address the need to measure the current brain sex. The score estimation method relies on the deep learning algorithm that integrates the feature extraction and classification processes into a single automated architecture. The method uses novel algorithms for scalp electroencephalogram data augmentation and channel rolling, allowing the claimed network to be more precise and converge faster. A SAAS-based framework for brain sex phenotype score calculation is disclosed in one or more embodiments.

### FIELD OF THE INVENTION

Embodiments of the present invention comprise methods and systems for service and method for brain sex phenotype score prediction on raw scalp electroencephalography (EEG).

### BACKGROUND OF THE INVENTION

The current neuroscientific developments are aiming towards a more personalized approach in identifying and treating mental disorders. However, personalization requires an additional understanding of the differences between the human sexes in its morphology and function. The human brain differs in males and females, which affects the frequency of the disease, detection of the correct symptomatology, age of onset, and treatment it requires. Identifying those differences may assist with uncovering the factors behind the variability that sex differences cause to the brain. Moreover, in order to gain insight into the nature and effects of sex differences, it is crucial to determine where they are located in the human brain.

According to several meta-analyses, the brain differs structurally in males and females on a number of levels. For instance, males tend to have higher raw volumes, raw surface areas, and white matter fractional anisotropy in the diffusion imaging. In contrast, females have higher raw cortical thickness and higher white matter complexity (Ritchie et al., 2018). Further, this accounts for the sex-related asymmetry in certain regions of the brain. For example, it was found that males have higher volume and tissue density in the right amygdala, hippocampus, putamen, temporal lobe, etc. In comparison, results of the female brains showed higher volume and density in the regions like the right inferior frontal gyms, middle frontal gyms, and frontal lobe, etc. Those differences have been previously linked to the genetic influence due to the brain's measure of high heritability.

Furthermore, these structural differences account for sex-associated differences in cognitive domains. Overall, females outperform males in language processing, such as verbal fluency, accuracy, and perceptual speed. At the same time, males exhibit advantages in working and spatial memory. Notably, female cognitive abilities' fluctuations are closely tied to the menstrual cycle (MC). The cognitive function dependency is associated with the changes in oestrogen and progesterone hormonal levels throughout the cycle. The cognitive peak occurs at ovulation when oestrogen is at its highest and progesterone is at stable low. Further, Kurth et al. (2021) point out that the sex differences associated with hormones become even more apparent and evolve throughout the lifespan. McCarthy et al. (2012) claim that the differences in the structure of the brain can be observed in children of pre-pubertal age. These differences occur as early as the neonate stage of human development. It is important to note that both sexes have all classes of reproductive hormones, albeit produced at different levels. Additionally, the listed above regions in the brain, certain cognitive dysfunctions and hormones are known to be sex-biased in neuropsychiatric disorders.

According to Smith et al. (2016) the prevalence of internalizing psychopathologies, such as depression and anxiety, can be classified as predominantly female. Whereas externalizing psychopathologies, such as ADHD, ODD, and CD, show higher instances in males. Previous studies suggest that this is influenced by the biological and socio-cultural factors combined. Moreover, it is not only the prevalence of the disorder that varies between the two sexes, but also the symptomatology differs substantially. For instance, statistically males and females are faced with equal risks for developing schizophrenia (Hartung & Widiger, 1998). However, the age of onset, symptoms, and disease severity varies between the two sexes. Males tend to develop schizophrenia up to 5 years earlier than females. In addition, the symptomatology differs significantly, while men more often present the “negative” symptoms, such as substance abuse and social withdrawal. On the other hand, females are more inclined to show mood disturbances, affective and depressive symptoms.

Previously, females have been largely excluded from clinical studies due to concerns over teratogenic effects. There is an existing sex-sensitive bias in neuropharmacology, which has led to inequality in researching possible avert effects and the overall efficacy of medication among the female population (Salmien et al., 2020). Females are twice as likely to develop depression, when compared to males. Recent studies showed that males and females have different responses and outcomes to antidepressant therapies. For example, females responded better to the selective serotonin reuptake inhibitors (SSRIs) and monoamine oxidase inhibitors (MAOIs) classes of antidepressants, and males to the tricyclic antidepressants (TCAs) (Sramek et al., 2016). This is mostly observed in women of child-bearing age and is attributed to the presence of oestrogen hormone.

Majority of the research explained above mostly looked at the existing differences through the biological prism. It was considered that psychopathology depends primarily on the chromosomes alignment in males (XY) and females (XX). These studies credited the classical view of binary brain sex representation, which would not explain why the differences exist, but not in all of the individuals. The binary in this view means that female and male brains are either the same or totally different. However, several studies argue that the sex variable in the brain acts as a continuous measure that varies from person to person.

One of the recent propositions in mental health research is to look at brain sex from a different perspective. Phillips et al. (2019) suggested considering that biological sex and brain sex may differ; in other words, the brain may simultaneously consist of female and male characteristics. One possible explanation may be that the sex of the brain goes beyond the classical biological concept and develops its own phenotype. Previously, McCarthy et al. (2011) suggested that it is not only that biological sex acts as a precursor for hormonal and cellular differences, but it is also the combination of environmental factors influencing the biology. The meta-analysis performed by Joel et al. (2015), have analyzed 5,500 MRI scans in order to uncover if the brains are sexually dimorphic. They separated the areas into “female-zone” and “male-zone”. Their findings revealed huge overlap in the majority of the brain areas of interest, forming the so-called “mosaics” of feminine-masculine traits in each individual. They suggested that the environment, such as nurture and culture may account for a major part of how the brain sex is being shaped. This brings novelty and provides an epigenetic view of how the human brain works as well as why some people are more prone to experience mental health issues than the others.

The main focus of brain sex phenotype prediction score is a Major Depressive Disorder (MDD); it is often presented with several co-morbid mental illnesses. Those are obsessive-compulsive disorder (OCD), bipolar disorder, attention deficit hyperactivity disorder (ADHD), anxiety spectrum disorders (panic disorder, generalized anxiety disorder), post-traumatic stress disorder (PTSD), and substance abuse disorders. Comorbidity can be defined as co-existing disorders in a specific individual for a prolonged period. MDD is classified by a core set of symptoms like prolonged low mood, anhedonia, changes in appetite, and sleep disturbances. Generally, females are more likely to be diagnosed with depression with a 2:1 ratio when compared to males. Additionally, MDD in females is often accompanied by co-morbid anxiety spectrum disorders, classified as internalizing psychopathology. Whereas depression in males presented with co-morbid ADHD and substance abuse, which is attributed to externalizing psychopathology (McCarthy et al., 2011).

The socio-cultural and hormonal influences partially cover this. Where socio-cultural influences may account for males being less likely to admit having any MDD symptomatology. This, in turn, may lead to substance abuse and worsening of the condition. However, the symptoms may vary from person to person and mainly depend on individual factors. The meta-analysis performed by Phillips et al. (2019) claims that biological sex is not always the case when it comes to psychopathology. Considering that brains are not dimorphic, a significant extent of features of one sex affect sex-biased variance in psychopathology. Therefore, it is safe to assume that an individual who is biologically male but has a distinctive female brain sex phenotype, and vice versa, will require a different approach taken for diagnosis and treatment of the condition.

To conclude, whether male- or female-born, each person does not necessarily abide by biologically predetermined sex when it comes to brain. Brainify.ai aims to use the brain sex phenotype (BSP) as a biomarker and provide a personalized approach towards identifying, diagnosing, and providing appropriate treatment for an individual with mental disorder by applying scalp electroencephalogram (EEG) technique combined with a deep learning approach.

To the best of our knowledge, van Putten et al. (2018) report remains the only successful attempt to automatically discriminate biological sex from clinical quality EEG data on adult subjects by using deep learning algorithms with the resulting classification accuracy above 80%. The remaining prior art is based on the manually selected features from time and frequency domains, and nonlinear features. It used statistical measures like variance, skewness, and kurtosis as time domain features. In some instances, the prior art calculated the spectral power of the EEG signals for frequency domain analysis. Based on the selected features, a prediction scheme that predicts the subject's sex is implemented using traditional machine learning algorithms like Logistic Regression, Support Vector Machine etc., rather than deep learning techniques.

Deep learning algorithms achieved great success inmultiple classification problems for various applications likecomputer vision and speech recognition. Some prior art utilized deep learning techniques to solve different neurophysiological problems based on raw scalp encephalography.

Motivated by these challenges and due to the significance of the non-binary patient-specific brain sex phenotype score prediction, thecurrent invention develops deep learning algorithms that combine the feature extraction and classification stages into a single automated framework and achieve accuracy not less than 85% on sex prediction on an adult subject.

### SUMMARY OF THE INVENTION

A novel patient-specific BSP score prediction technique based on deep learning and applied to long-term scalp EEG recordings is provided herein.

A patient-specific brain sex phenotype score is defined as a difference between algorithmically predicted brain sex probability and genetic sex.

A brain sex phenotype score prediction algorithm based on deep learning that integrates the feature extraction and classification processes into a single automated architecture is claimed herein.

The method uses novel algorithms for raw scalp electroencephalogram data augmentation and channel rolling to achieve better model quality.

The deep learning-based algorithm, data augmentation, and channel rolling algorithms are implemented as a cloud-based service.

### BRIEF DESCRIPTION OF THE DRAWING FIGURES

The patent or application file contains at least one drawing executed in color. Copies of this patent or patent application publication with color drawing(s) will be provided by the Office upon request and payment of the necessary fee.

FIG. 1 is showing a system for EEG electrodes position sites (electrode placement map);

FIG. 2 is a flow diagram illustrating workflow based on one of the datasets consisting of 1094 EEG sessions (data flow through the service);

FIG. 3 is a diagram showing optimal segment size selection, the segment size affecting the number of artifact-free segments available for training and the final accuracy of the model;

FIG. 4 is a diagram showing the “channel rolling and shifting” method;

FIG. 5 is a diagram showing an architecture as a SAAS framework (solution architecture);

FIG. 6 is a diagram showing a deep convolutional neural networks (CNN) architecture overview; and

FIG. 7 is a diagram showing a cross-validation scheme consisting of 10 iterations, at each iteration, 10% of data is used for testing, 10% for tuning, and 80% for training.

### DETAILED DESCRIPTION OF THE PREFERRED EMBODIMENTS

The subject matter of the present invention is described with specificity herein to meet statutory requirements. However, the description itself is not intended to limit the scope of claims necessarily.

To calculate the BSP score, the electroencephalograph is deployed. The electroencephalogram is a non-invasive tool used to measure the brain's activity. The measurement is recorded through the electrodes attached to the individual's scalp. EEG is a relatively fast method to administer temporal resolution with high precision in resting-state brain activity. Moreover, it is cost-effective and allows to perform a measurement in a natural setting, unlike most other tools. EEG is currently widely used for research on brain-computer interfaces (BCI) due to its portability and other features explained above (Lawhern, et al., 2018; Uran et al., 2019). Moreover, EEG is safer than PET, as it does not involve radiation in its screening technique. It is not metal-sensitive like MRI, and unlike genetic and serum chemistry, it directly measures brain activity. EEG is promising for acquiring steady raw data. It operates on recordings of the wavelengths of the specific bands. Those are an alpha band (8-12 Hz), a beta band (13-38 Hz), delta band (1-4 Hz), theta band (4-8 Hz), and gamma band (<30 Hz).

There was used a 10-10 system for EEG electrodes position sites as represented in FIG. 1. The electrode placements are labelled according to the lobes of the brain (Fp—pre-frontal, F-frontal, C— central, P— parietal, O— occipital, T— temporal). Ground (GND) electrodes are placed on the forehead and used to prevent power noise from interfering with small biopotential signals of interest. AFz, Fz, Cz, Pz electrodes are placed over the corpus callosum area and used for reference points. A1, A2 electrodes used for contralateral referencing across all the EEG electrodes.

It is vital to measure the resting-state brain activity to denote intrinsic neural activity, which is not task-dependent. Resting-state connectivity can be defined as a considerably correlated activity between functionally related brain regions without any stimulus/task. Due to the existence of resting-state connectivity in visual cortices. There were used both conditions, eyes-closed/eyes-opened, in the resting-state EEG recording in order to establish the baseline performances. MDD is known for the alteration across the EEG patterns. The identification of those alterations allowed the EEG prediction of the outcome in antidepressant therapies. However, the sex differences are rarely considered despite its association with the EEG alterations in the response to certain SSRIs and SNRIs medications (Arns, et al., 2016; Wade and Iosifescu., 2016; Jaworska et al., 2019). Moreover, the brain sex is not taken onto account either. Though, as it was mentioned above, it is possible to predict the brain sex phenotype from the EEG data by using the deep convolutional neural network technique (van Putten, 2018).

In the current diagnostic climate, patients may spend years seeking the appropriate options for correct diagnosis and treatment of the condition. Identifying specific biomarkers such as brain sex phenotype shall provide additional aid in the successful diagnosis and will increase the effectiveness of remission onset. Therefore, BSP will be used for a more accurate diagnosis of MDD and co-morbid disorders as well as to predict the appropriate treatment. It is expected that BSP score equal to 0 will indicate that the sex of the brain matches the biological sex of the individual. If the BSP score>0, then the brain sex of a biological male has female phenotype. The level of expression depends on the score's proximity to 1. Wherein, if BSP score<0, then the biological female has the male brain sex phenotype, the level of expression depends on score's proximity to −1.

Since a way to measure effective BSP is unknown, it was invented a novel method for its estimation. The idea is to use a predicted sex class probability of being a male taken from the output of a deep learning classifier as a proxy for an actual brain sex phenotype. Based on this idea, the BSP score is to be defined as the difference between a genetic (biological) sex and a predicted brain sex probability. According to the above BSP score definition, its prediction problem can be reduced to a continuous sex prediction task, which, in turn, is solved as a part of the binary classification task of the genetic sex of a subject. In short, the deep model is trained as a binary classifier, then the raw predicted probability of sex is used to calculate the BSP score.

This invention also provides a novel way to automatically extract essential features from raw EEG recordings developing deep learning algorithms without manual preprocessing. Deep Convolutional Neural Network (DNN) is used to learn the discriminative time and spatial features from the raw EEG recordings, and Single-Layer Perceptron is applied to solve the classification task.

Although there is a fair amount of prior art focused on various classification tasks based on resting state EEG recordings cut into segments, there is no standard duration for them. The length of an EEG segment to be an atomic piece of information for training a deep learning model is experimentally selected to be 4 seconds.

DNNs are known to learn worse due to a well-known “small receptive field” problem. To address it, it is proposed the inventive “channel rolling” method, which extends the receptive field of the first convolutional layer of the network to all input EEG channels.

Deep learning models are prone to overfit when trained on insufficiently large datasets. This invention demonstrates an algorithm for data augmentation that adapts the well-known image processing approaches to raw EEG recordings processing to address this issue.

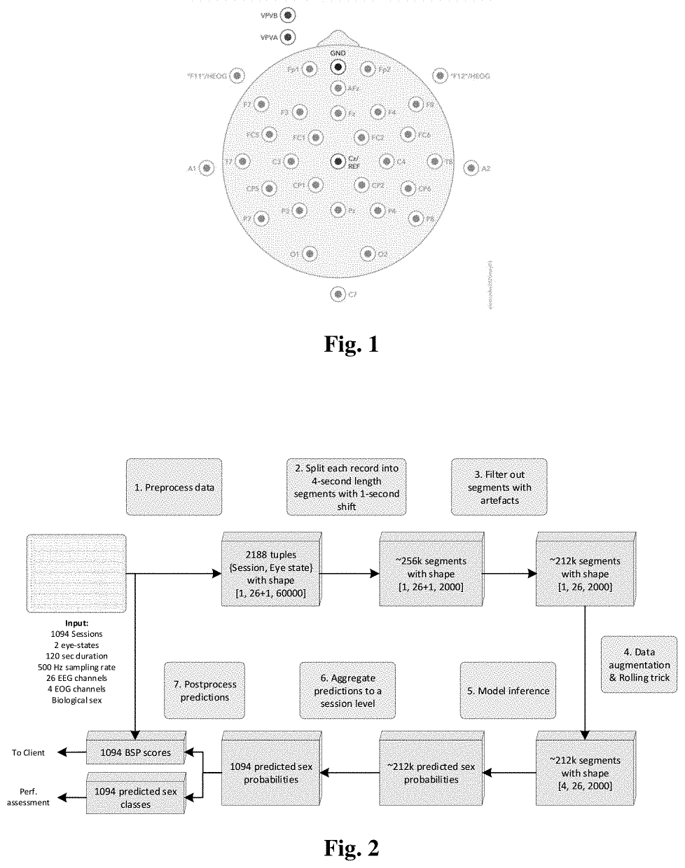

A Software as a service (SAAS) framework for accurate BSP score prediction suitable for real-time operation is incorporated in one or more embodiments, as depicted in FIG. 2 (Examples). The client software collects resting state scalp EEG and sends it to the cloud-based Service. The Service processes the request in real-time mode and returns the resulting BSP score.

For the purpose of understanding the SERVICE AND METHOD FOR BRAIN SEX PHENOTYPE SCORE PREDICTION ON RAW SCALP EEG, references are made in the text and supplementary materials to exemplary embodiments of a SERVICE AND METHOD FOR BRAIN SEX PHENOTYPE SCORE PREDICTION ON RAW SCALP EEG, only some of which are described herein. It should be understood that no limitations on the scope of the invention are intended by describing these exemplary embodiments. One of ordinary skill in the art will readily appreciate that alternate, but functionally equivalent components, materials, designs, and equipment may be used. The inclusion of additional elements may be deemed readily apparent and obvious to one of ordinary skill in the art. Specific elements disclosed herein are not to be interpreted as limiting but rather as a basis for the claims and as a representative basis for teaching one of ordinary skill in the art to employ the present invention.

### EXAMPLES

### Example 1

The EEG recording is collected via one of the commercially available EEG recording solutions. The client-side software is responsible for providing biological sex of a subject and a 26-channels EEG-recording based on the 10-10 electrode international system with linked mastoids montage containing the following EEG channels: Fp1, Fp2, F7, F3, Fz, F4, F8, FC3, FCz, FC4, T7, C3, Cz, C4, T8, CP3, CPz, CP4, P7, P3, Pz, P4, P8, 01, Oz, 02, as well as the following EOG channels: Vertical positive vertical above, Vertical negative vertical below, Horizontal left, Horizontal right. Then, the client-side software transmits all mentioned data to the invented Service via its REST API.

The client is responsible for the provision of EEG data assessed during resting state, consisting of: a 2-minute Eyes Open (EO) task, where the subject is asked to rest quietly, with eyes open and focus on a dot at the center of the computer screen in front of them, and a 2-minute Eyes Closed (EC) task, where the subject is asked to close their eyes and retain the same position as before. Vertical- and horizontal eye movements should be recorded with electrodes placed 3 mm above the left eyebrows and 1.5 cm below the left bottom eye-lid, and 1.5 cm lateral to the outer canthus of each eye, respectively.

The workflow consists of several steps, which will be considered in detail further: data preprocessing, EEG segmentation, artifact removal, data augmentation and channel rolling trick, model inference, predictions aggregation, and predictions postprocessing. FIG. 2 illustrates this workflow based on one of the datasets consisting of 1094 EEG sessions with 2 minutes of recording with open and closed eyes each.

1. Data Preprocessing

Data preprocessing is required to remove unnecessary artifacts (such as eye blinks) from the training data. To preprocess and de-artifact large amounts of EEG datasets, the previously known art of automatic preprocessing routines (Brainclinics resources, 2022) was adapted within the Service. In short, the bipolar EOG is computed and removed from the EEG signal using the method proposed by Gratton et. al., 1983. Data is demeaned and bandpass-filtered between 0.5 to 100 Hz, and the notch-frequency of 50 Hz or 60 Hz is removed. Extra ‘artifacts’ channel is added to the data with a flag whether various artifact signals were detected or not based on detection of: 1) electromyography (EMG) activity, 2) sharp channel-jumps (up and down), 3) kurtosis, 4) extreme voltage swing, 5) residual eye blinks, 6) electrode bridging (Alschuler et al., 2014), and 7) extreme correlations.

2. EEG Segmentation

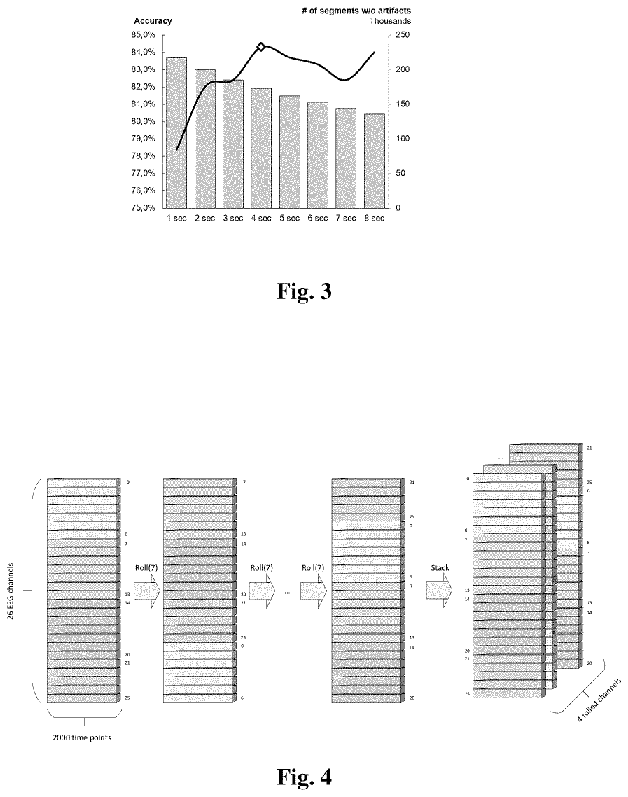

Resting state EEG typically has a length of several minutes, and for later usage in DL training is often split into shorter pieces with a span of several seconds. The decision to use only artifact-free data limits possible segment length because the number of artifact-free segments decreases while their length increases. Optimal EEG segment length of 4 seconds was found experimentally, as shown in FIG. 3.

3. Artifact Removal

An EEG segment containing at least one time-point with artificial data is considered invalid and is eliminated from the output dataset. Artifacts in raw EEG recordings are removed from the training data to force the model to focus on intrinsic brain activity.

4. Data Augmentation and Channel Rolling

By this invention a specific training method for brain sex prediction is demonstrated, which method consists of the data augmentation technique and the channel rolling described below.

Data augmentation is a well-established technique in image processing deep learning applications, but previous deep learning solutions did not use it for raw EEG processing. The training method for claimed deep CNN involves usage of one of the data augmentation methods for each EEG segment during the training process:
with a probability of 50%, apply gaussian noise to the input tensor with random standard deviation drawn from a uniform distribution (0,1] μV.
with a probability of 70%, apply random dropout of Bₖ consequent time-points in K EEG channels the input tensor data, where K and Bₖ are drawn from uniform distributions [1, 8] and [1, 1800], respectively.
with a probability of 50%, apply random amplification of the input tensor with a multiplier M_{ch} drawn from a uniform distribution [0.8, 1.2] for each EEG channel ch.
With a probability of 50%, shrink or stretch time axis with a factor uniform distribution [0.8, 1.2].
With a probability of 50%, inverse time flow for all EEG channels.

One of the main features of deep learning convolutional neural networks is the repeated use of the convolution operation: the convolution of the first layer is applied to the input data, the convolution of the 2nd layer to the output of the 1st layer, etc. The receptive field specifies which region of the input the convolution operation will process to get one point in the output.

The “channel rolling” method is claimed to extend the receptive field of the first convolutional layer to all 26 EEG channels. For this, the input tensor is shifted by seven along the EEG channels dimension (dimension #1) 3 times in a turn. The resulting tensors are then stacked with the original tensor along dimension #0. After this operation, the original tensor with dimensions [1, 26, 2000] turns into a tensor with dimensions [4, 26, 2000], as illustrated in FIG. 4.

5. Model Inference

The invention provides the deep learning model for accurate brain sex prediction. the Sex Prediction Problem is Formulated as a Binary Classification Task (0-Female, 1—Male). The model receives an EEG segment as an input and outputs a real number in the range [0, 1], representing probability for an input segment to belong to a session of a male subject.

6. Predictions Aggregation

The model outputs predicted sex probability for a set of segments for each session (1). These probabilities are averaged over segments for both eye states to obtain a session-level sex probability (2).

$${Proba}_{s}^{i} ∈ [ 0 , 1 ] , i = 1 … {N}_{s} , {N}_{s} = number of segments in \\ session s for both eye states \qquad ( 1 )$$

$${Proba}_{s} = \frac{{\sum }_{i = 1}^{{N}_{s}} {Proba}_{s}^{i}}{{N}_{s}} \qquad ( 2 )$$

7. Predictions Postprocessing

The resulting BSP score is calculated as defined by (3).

[symbol]

=Probaₛ−yₛ^{true} (3)

where yₛ^{true} is the biological sex of a subject.

Also, the predicted brain sex probability at the session level is binarized to a predicted sex class {0, 1} as described in (4), which is needed to assess the model performance

Ŷₛ=Probaₛ≥0.5 (4)

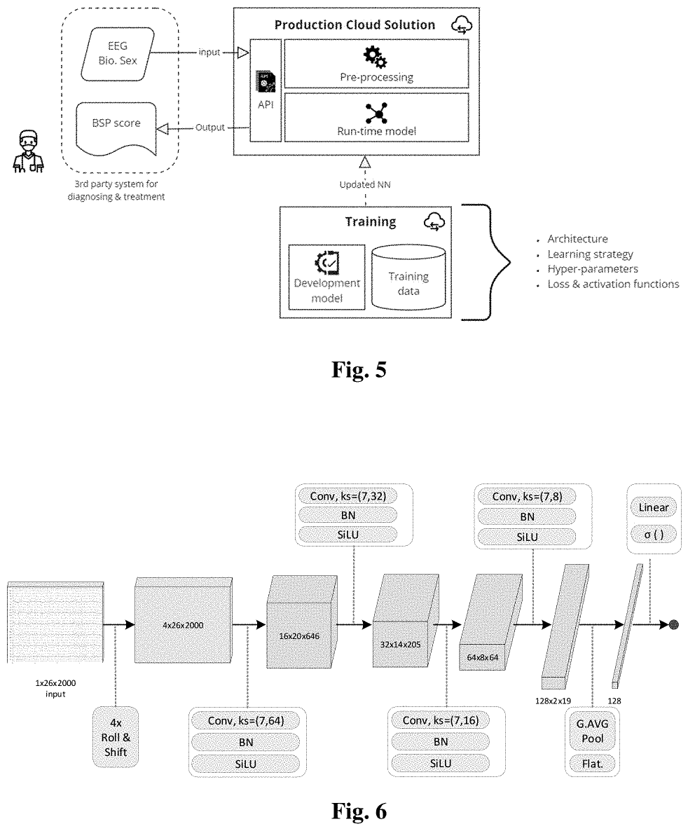

The workflow depicted above is implemented as a SAAS framework. FIG. 5 details the architecture of this framework.

Key Components of the SaaS Framework are:
Production cloud solution is standalone SaaS platform that consists of the following modules:
   Preprocessing module of the framework comprises workflow steps 1, 2 and 3.
   Run-time (trained) model module of the framework realizes BSP calculation and prediction logic (steps 5, 6 and 7).
   API to expose the resulting BSP score to 3rd party systems.
Training solution is used for training and improving the run-time model. It consists of:
   Development model. This module also includes model architecture, learning strategy, loss & activation functions and other parameters required to develop a predictive model.
   Training data.
Client 3rd party systems for diagnosing & treatment will use REST API of Production Cloud Solution to obtain the predicted BSP score. The incoming EEG recording will be preprocessed and further analyzed by the deep CNN on the Service side by Production cloud solution.

Integrations:
Integration between Production cloud solution and Training solution to allow updating production run-time model with changes from the Training environment as the model continues to learn and improve.

### Example 2

Convolutional Neural networks (CNNs) have shown great success in different pattern recognition and computer vision applications. This is due to the ability of CNN to automatically extract significant spatial features that best represent the data from its raw form without any preprocessing and without any human decision in selecting these features. The sparse connectivity and parameter sharing of CNN give it high superiority regarding the memory footprint as it requires much less memory to store the sparse weights. The equivariant representation property of the CNN increases the detection accuracy of a pattern when it exists in a different location across the input signal. A typical CNN comprises three types of layers: convolution layer, pooling layer, and fully connected layer. The convolution layer is used to generate the feature map by applying filters with trainable weights to the input data. This feature map is then down-sampled by applying the pooling layer to reduce the features' dimension and, therefore, the computational complexity. Finally, the fully connected layer is applied to all the preceding layer's output to generate the one-dimensional feature vector. CNN is used as a feature extractor to replace the complex feature engineering used in the prior art.

The claimed deep CNN architecture model is shown in FIG. 6, in which the EEG segment is converted into a 3D matrix to be suitable for deep CNN. The channel rolling operation represents the input part of the network. The middle part consists of four convolutional blocks. Each convolutional block contains Convolution operation (5) followed by Batch Normalization and activation function. Parameters of operations for each convolutional block are represented in the Table 1.

$${Conv}_{ch , t} = {\sum }_{m = - ∞}^{∞} {\sum }_{n = - ∞}^{∞} {\sum }_{c = 1}^{{N}_{c}} W [ c , m , n ] * I [ c , ch - m , t - n ] , \qquad ( 5 )$$

where c is an input tensor channel, ch is an EEG channel, t is a time-point.

TABLE 1
Convolution blocks of the deep CNN claimed.

| Convolution | Convolution parameters | | | Batch | Activation |
|---|---|---|---|---|---|
| Block | Kernels | Kernel size | Stride | normalization | function |
| conv-1 | 16 | (7, 64) | (1, 3) | Yes | SiLU |
| conv-2 | 32 | (7, 32) | | | (aka |
| conv-3 | 64 | (7, 16) | | | “swish-1”) |
| conv-4 | 128 | (7, 8) | | | |

The batch Normalization technique is used to improve the training convergence and reduce overfitting. Both the input and output of a Batch Normalization layer are four-dimensional tensors, which refers to as I_{b,c,ch,t} and BN_{b,c,ch,t}, where b corresponds to examples within a mini-batch. Batch Normalization applies the same normalization for all activations in a given channel,

$${BN}_{b , c , ch , t} = {γ}_{c} \frac{{I}_{b , c , ch , t} - {μ}_{c}}{\sqrt{{σ}_{c}^{2} + ε}} + {β}_{c} , ∀ b , c , ch , t . \qquad ( 6 )$$

Here, Batch Normalization subtracts the mean activation

$${μ}_{c} = \frac{1}{\vert B \vert} {\sum }_{b , ch , t} {I}_{b , c , ch , t}$$

from all input activations in channel c, where B contains all activations in channel c across all features b in the entire mini-batch and all “spatial” (ch, t) locations. Subsequently, Batch Normalization divides the centered activation by the standard deviation σ_{c}. (plus ε for numerical stability) which is calculated analogously. During testing, running averages of the mean and variances are used. Normalization is followed by a channel-wise affine transformation parametrized through γ_{c}, β_{c}, which are learned during training.

The Sigmoid-weighted Linear Unit (SiLU) activation function, as defined by (7), is used across the convolution layers to add nonlinearity, ensure robustness against noise in the input data, and achieve faster backpropagation convergence.

$$silu ( x ) = x * σ ( x ) \qquad ( 7 )$$

$$σ ( x ) = \frac{1}{1 + {e}^{- x}} \qquad ( 8 )$$

The final part of deep CNN consists of a Global Average Pooling (9) layer and a single Linear (10) layer, also referenced as a Single-Layer Perceptron, of size 128 and Sigmoid activation function (8), which are responsible for the sex probability prediction.

$${GlobalAVGPoolI}_{b , c} = \frac{1}{{N}_{ch} * {N}_{t}} {\sum }_{m = 0}^{{N}_{ch}} {\sum }_{n = 0}^{{N}_{t}} {I}_{b , c , m , {n}^{′}} \qquad ( 9 )$$

where N_{ch} and Nₜ are sizes of input tensor across EEG channel and time dimensions, respectively.

Linear_{b}=W*I_{b}+b₀, (10)

where W and bₒ are learned parameters.

The loss function used is the binary cross-entropy defined by (11)

bce(y,ŷ)=−(y*log log(ŷ)+(1−y)*log log(1−ŷ)) (11)

where ŷ and y are predicted and target classes, respectively.

The model is trained with backpropagation using Adam optimization algorithm with starting learning rate 3e-5, reduce-on-plateau scheduler with the patience of three epochs, and early stopping after ten epochs without validation metric improvement.

### Example 3

The model's performance is evaluated on the brain sex classification task by calculating binary accuracy metric (12)-(16) on the TD-BRAIN and TUH EEG Corpus datasets based on EEG recordings for subjects of age 18 years and older.

$$TP = {\sum }_{s} \hat{{Y}_{s}} == {Y}_{s} , where {Y}_{s}^{true} == male \qquad ( 12 )$$

$$TN = {\sum }_{s} \hat{{Y}_{s}} == {Y}_{s} , where {Y}_{s}^{true} == female \qquad ( 13 )$$

$$FP = {\sum }_{s} \hat{{Y}_{s}} != {Y}_{s} , where {Y}_{s}^{true} == male \qquad ( 14 )$$

$$FN = {\sum }_{s} \hat{{Y}_{s}} != {Y}_{s} , where {Y}_{s}^{true} == female \qquad ( 15 )$$

$$Accuracy = \frac{TP + TN}{TP + TN + FP + FN} \qquad ( 16 )$$

Finally, the binary accuracy metric at the session level is calculated.

The Two Decades-Brainclinics Research Archive for Insights in Neurophysiology (TD-BRAIN) EEG database by Brainclinics Foundation (Brainclinics resources, 2022) clinical lifespan database (mean age 44.98 (15.35 SD) years, range 18-85 years) contains resting-state eyes open and eyes closed, raw EEG-data complemented with relevant clinical and demographic data of a heterogeneous collection of 1094 sessions from 1030 psychiatric patients collected between 2001 to 2021. Sixty subjects have more than one session (2 or 3). Since between sessions of one subject, a significant time elapses from 47 days to 14 years (mean 1.22 (1.95 SD) yrs.), such sessions are considered independent. The quality metric of the model is calculated at the session level. The distribution of men and women is equal (547 sessions each). The main formal diagnoses included are Major Depressive Disorder (MDD; N=198), Attention Deficit Hyperactivity Disorder (ADHD; N=144), and Obsessive-Compulsive Disorder (OCD; N=56).

TUH EEG Corpus (Obeid and Picone, 2016) contains clinical EEG data collected from archival records at Temple University Hospital (TUH). The completed corpus comprises 16,986 sessions from 10,874 unique subjects. Each of these sessions contains at least one EDF file and one physician report. Subjects were 51% female and ranged in age from less than 1 year to over 90 (average 51.6, 55.9 SD). A small subset of the corpus containing 2530 outpatient sessions of length not less than 10 minutes is used to assess the model performance. The corpus doesn't offer structured metadata, e.g., patient's age. For this reason, the patient's age and other statistics of the selected subset are not provided here.

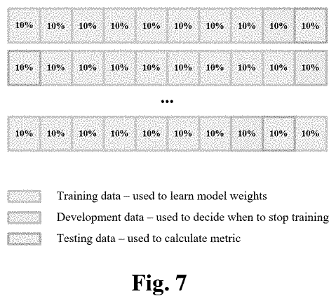

10-fold cross-subject cross-validation is used to calculate the model performance metrics. The training consists of 10 iterations, as shown in FIG. 7 and described below.

At each iteration, a fold containing 10% of all data is used for testing, resulting in all data being tested after ten cycles. The proposed validation strategy provides the most realistic performance metric evaluation on a given dataset because each EEG session is tested. The model's training process utilizes learning rate scheduling techniques such as “reduce on plateau” and “early stopping”. These techniques require an independent development (tuning) dataset formed from another fold. The remaining eight folds are used for training purposes.

TABLE 2
The model performance on the different datasets

| | | TUH | TD-BRAIN |
|---|---|---|---|
| Dataset properties | N | 2530 | 1094 |
| | Age | unknown | 18-85 years |
| | | | mean 44.98 (15.35 SD) |
| | Males | 43% | 50% |
| Accuracy of brain | | 85% | 85% |
| sex class prediction | | | |

The achieved results show that the claimed method is reliable, efficient, and suitable for real-time application to predict brain sex phenotype score and accompanying brain sex class based on raw resting state EEG data. The high quality of 85% for brain sex class prediction and speed<10 ms make the claimed service a good choice for a smart mental health-care system to improve the quality of mental disorders diagnostic and treatment selection.

1. A patient-specific brain sex phenotype score prediction method, the method comprising defining a patient-specific brain sex phenotype score as a difference between algorithmically predicted brain sex probability and genetic sex: [symbol]
=Probaₛ−yₛ^{true},
where Probaₛ ∈ [0,1] is the predicted sex and yₛ^{true} ∈ {0,1} is the biological sex of a subject, wherein:
a BSP score is equal to 0 is indicative that the sex of the brain matches the biological sex of the individual;
the BSP score>0 is indicative that the brain sex of a biological male has female phenotype, the level of expression depending on the score's proximity to 1;
the BSP score<0 is indicative that the biological female has the male brain sex phenotype, the level of expression depending on score's proximity to −1.

2. A patient-specific brain sex phenotype score prediction method comprising deep learning algorithms, wherein said prediction method does not include hand-crafted features obtained from scalp electroencephalogram recordings.

3. The method of claim 2 further comprising classification tasks, and wherein said classification tasks comprise the step of applying an artificial neural network to raw electroencephalogram recordings as a classifier.

4. The method of claim 3 wherein said artificial neural network is Single-layer Perceptron.

5. The method of claim 3 wherein said classification tasks further comprise the step of applying to said raw resting state electroencephalogram recordings a Deep Convolutional Neural Network to learn the discriminative time-spatial features between male and female brain states before said artificial neural network is applied for said classification.

6. A brain sex prediction deep neural network training method comprising data augmentation and channel rolling.

7. The method of claim 2 wherein said deep learning-based algorithm and the method of claim 6 wherein said data augmentation and channel rolling are implemented as a cloud-based service.

8. The data augmentation method of claim 6 is defined by the following algorithm:
with a probability of 50%, apply gaussian noise to the input tensor with random standard deviation drawn from a uniform distribution (0,1] μV.
with a probability of 70%, apply random dropout of Bₖ consequent time-points in K EEG channels the input tensor data, where K and Bₖ are drawn from uniform distributions [1, 8] and [1, Len_{seg}*SFreq*0.9], respectively, where Len_{seg} is the length of one EEG segment in seconds and SFreq is the sampling frequency of raw EEG data.
with a probability of 50%, apply random amplification of the input tensor with a multiplier M_{ch} drawn from a uniform distribution [0.8, 1.2] for each EEG channel ch.
With a probability of 50%, shrink or stretch time axis with a factor uniform distribution [0.8, 1.2].
With a probability of 50%, inverse time flow for all EEG channels.

9. The channel rolling method of claim 6 extends the receptive field of the first convolutional layer of the network to all input EEG channels and is defined by the following algorithm:
xᵢₙ-the input tensor with shape(xᵢₙ)=[1, N_{channels}, Len_{seg}*SFreq], where Len_{seg} is the length of one EEG segment in seconds and SFreq is the sampling frequency of raw EEG data.
$${N}_{steps} = ⌈ \frac{{N}_{channels}}{{KernelSize}_{channels}} ⌉ ,$$
the resulting number of channels in the output tensor dimension #0, where KernelSize_{channels} is the size of the kernel of the first convolutional layer for the EEG channels dimension.
xₒᵤₜ=xᵢₙ, xᵢ=xᵢₙ
for i in [2..Nₛₜₑₚₛ]:
i. xᵢ=roll(xᵢ,KernelSize,1)—roll tensor xᵢ by KernelSize shifts along EEG channels dimension #1.
ii. xₒᵤₜ=concatenate (xₒᵤₜ, xᵢ, 0)-concatenate tensors xₒᵤₜ and xᵢ in the dimension #0.
return xₒᵤₜ

10. The Deep Convolutional Neural Network of claim 5 having the following architecture:
Conv(kernelsize=(KS₀, KS₁), kernels=16, stride=(1, Strideₜᵢₘₑ))
BatchNorm( )
Activation( )
$$Conv ( kernelsize = ( {KS}_{0} , ⌊ \frac{{KS}_{1}}{2} ⌋ ) , \\ kernels = 32 , stride = ( 1 , {Stride}_{time} ) )$$
BatchNorm( )
Activation( )
$$Conv ( kernelsize = ( {KS}_{0} , ⌊ \frac{{KS}_{1}}{4} ⌋ ) , \\ kernels = 64 , stride = ( 1 , {Stride}_{time} ) )$$
BatchNorm( )
Activation( )
$$Conv ( kernelsize = ( {KS}_{0} , ⌊ \frac{{KS}_{1}}{8} ⌋ ) , \\ kernels = 128 , stride = ( 1 , {Stride}_{time} ) )$$
BatchNorm( )
Activation( )
GlobalAVGPool( )
Linear(in=128, out=1)
Sigmoid( )
Where:
the kernel size KS⁰ takes one value from the range [2, 10];
KS¹ takes one value from the range [[SFreq/10], SFreq], where SFreq is the sampling frequency of raw EEG data;
stride step along time axis Strideₜᵢₘₑ takes one value from the range [3, 10].
